(19) United States
(12) Patent Application Publication
(10) Pub. No.: US 2023/0108741 A1
(43) Pub. Date: Apr. 6, 2023

(54) INSERTION DEVICE AND OPERATION PORTION UNIT

(71) Applicant: OLYMPUS MEDICAL SYSTEMS CORP., Tokyo (JP)

(72) Inventors: Takumi KAWAKAMI, Tokyo (JP); Eijiro SATO, Tokyo (JP)

(73) Assignee: OLYMPUS MEDICAL SYSTEMS CORP., Tokyo (JP)

(21) Appl. No.: 17/958,492

(22) Filed: Oct. 3, 2022

Related U.S. Application Data
(60) Provisional application No. 63/251,912, filed on Oct. 4, 2021.

(51) Int. Cl. A61B 1/005

(52) U.S. Cl. CPC A61B 1/0052; A61B 1/0057; A61B 1/00119

(57) ABSTRACT

Insertion device according to an aspect of the present disclosure includes: an insertion device, comprising an insertion portion configured to be inserted into a subject. At least one traction member configured to pull a distal end side portion of the insertion portion. An operation portion provided on a proximal end side of the insertion portion. At least one sheath member inserted through the insertion portion, wherein the at least one traction member is inserted through an inner channel in the at least one sheath member. A rotating member to which one end of the at least one traction member is connected, the rotating member configured to rotate, with an external operation force, around a first axis of a first shaft member provided in the operation portion. At least one guide member disposed in the operation portion, wherein at least a portion of the at least one guide member is located on a first shaft member side with respect to a tangential line of an outer circumference of the rotating member, the tangential line passing through a point on an end of the at least one sheath member that is located within the operation portion. Wherein the at least one guide member is configured to guide the at least one traction member such that at least a portion of a path of the at least one traction member passes on the first shaft member side of the tangential line.

### RELATED APPLICATION DATA

This application is based on and claims priority under 37 U.S.C. § 119 to U.S. Provisional Application No. 63/251,912 filed on Oct. 4, 2021, the entire contents of which are incorporated herein by reference.

### FIELD OF THE DISCLOSURE

The present disclosure relates to insertion device such as an endoscope and an operation portion unit.

### BACKGROUND

There is insertion device such as an endoscope and a catheter inserted into a subject such as a patient. When the insertion device is, for example, an endoscope, an insertion portion of the endoscope includes a bending portion. A doctor, who is a user of the endoscope, operates a lever, a knob, or the like for bending operation when bending the bending portion. The doctor can bend the bending portion of the insertion portion in a desired direction by operating the lever, the knob, or the like provided in the operation portion.

More specifically, a plurality of bending operation wires are inserted through the insertion portion in order to bend the bending portion. By operating the lever, the knob, or the like, the plurality of (for example, two) bending operation wires inserted through the insertion portion are pulled or slacked. The plurality of bending operation wires are pulled or slacked, whereby the bending portion bends. The respective bending operation wires are inserted through a wire sheath (or a guide coil) provided in the insertion portion. An end portion of the wire sheath is disposed in the operation portion.

For example, as disclosed in International Publication No. 2017/043124, end portions of the bending operation wires extending from the end portion of the wire sheath are fixed to a pulley. A movement of the lever, the knob, or the like of the operation portion is converted into a rotation of the pulley. If the respective bending operation wires can be pulled more by a smaller amount of operation on the lever, the knob, or the like, operability for the user is improved. For example, when the user desires to further reduce a range of an operation angle of the lever, the operability can be improved by using a pulley having a larger diameter.

### SUMMARY OF THE DISCLOSURE

Insertion device according to an aspect of the present disclosure includes an insertion device comprising an insertion portion configured to be inserted into a subject. At least one traction member is configured to pull a distal end side portion of the insertion portion. An operation portion is provided on a proximal end side of the insertion portion. At least one sheath member is inserted through the insertion portion, wherein the at least one traction member is inserted through an inner channel in the at least one sheath member. One end of the at least one traction member is connected to a rotating member, the rotating member configured to rotate, with an external operation force, around a first axis of a first shaft member provided in the operation portion. At least one guide member is disposed in the operation portion. At least a portion of the at least one guide member is located on a first shaft member side with respect to a tangential line of an outer circumference of the rotating member, the tangential line passing through a point on an end of the at least one sheath member that is located within the operation portion. The at least one guide member is configured to guide the at least one traction member such that at least a portion of a path of the at least one traction member passes on the first shaft member side of the tangential line.

An operation portion unit according to an aspect of the present disclosure is an operation portion unit configured to fix to one end of at least one traction member configured to pull a distal end side portion of an insertion portion that is configured to be inserted into a subject portion, the operation portion unit comprising a housing. A rotating member is provided in the housing and configured to rotate around a first axis of a first shaft member by an external operation force. At least one guide member is disposed in the housing. At least a portion of the at least one guide member is located on a first shaft member side with respect to a tangential line of an outer circumference of the rotating member, the tangential line passing through a point on an end of the at least one sheath member. The at least one traction member extends from the at least one sheath member. The at least one guide member is configured to guide the at least one traction member such that at least a portion of a path of the at least one traction member passes on the first shaft member side of the tangential line.

Another aspect of the present disclosure is an insertion device comprising an insertion portion having a distal portion. A first wire is inserted into the insertion portion, the first wire having a first end and a second end, the first end connected to the distal portion. An operation portion is configured to pull the first wire and bend the distal portion. A shaft is placed inside the operation portion. A main pully is configured to guide the first wire and configured to rotate around the shaft. A first sheath is inserted into the insertion portion and receiving the first wire. The first sheath is configured to guide the first wire toward the shaft.

### BRIEF DESCRIPTION OF THE DRAWINGS

FIG. 1 is an exterior view of an endoscope in a first embodiment.

FIG. 2 is a diagram showing an internal structure of an operation portion in the first embodiment.

FIG. 3 is a perspective view of the operation portion showing a cross section of the operation portion taken along line in FIG. 2.

FIG. 4 is a schematic diagram for explaining movements of two bending operation wires corresponding to a rotation of a pulley in the first embodiment.

FIG. 5 is a diagram showing an internal structure of the operation portion according to a modification 1-1 of the first embodiment.

FIG. 6 is a perspective view of an inside of a housing member of the operation portion according to the modification 1-1 of the first embodiment.

FIG. 7 is a perspective view of a bearing according to the modification 1-1 of the first embodiment.

FIG. 8 is a perspective view of a guide unit according to the modification 1-1 of the first embodiment.

FIG. 9 is a side view of the guide unit in which a columnar portion is replaced with a pulley according to the modification 1-1 of the first embodiment.

FIG. 10 is a schematic sectional view of the operation portion according to a modification 1-2 of the first embodiment.

FIG. 11 is a schematic sectional view of the operation portion according to a modification 1-3 of the first embodiment.

FIG. 12 is a schematic diagram showing an internal structure of the operation portion according to a modification 1-4 of the first embodiment.

FIG. 13 is a schematic side view of the operation portion viewed in a direction parallel to an axis of a shaft member and orthogonal to a longitudinal axis of the operation portion according to the modification 1-4 of the first embodiment.

FIG. 14 is a diagram showing an internal structure of an operation portion in a second embodiment.

FIG. 15 is a perspective view of the operation portion showing a cross section of the operation portion taken along XV-XV line in FIG. 14.

FIG. 16 is a perspective view of an inside of a housing member of the operation portion according to a modification 2-1 of the second embodiment.

FIG. 17 is a perspective view of the operation portion showing a cross section of the operation portion taken along XVII-XVII line in FIG. 16.

FIG. 18 is a schematic diagram showing an internal structure of an operation portion showing disposition of a pulley and two guides according to a third embodiment.

FIG. 19 is a schematic side view of the operation portion showing disposition of the pulley and the two guides in the operation portion according to the third embodiment.

FIG. 20 is a schematic side view of the operation portion showing disposition of the guides long in a central axis direction and the pulley in the operation portion according to the third embodiment.

FIG. 21 is a schematic view for explaining movements of two bending operation wires corresponding to a rotation of the pulley according to the third embodiment.

FIG. 22 is a perspective view of an inside of a housing member of an operation portion according to a fourth embodiment.

FIG. 23 is a perspective view of the operation portion showing a cross section of the operation portion taken along XXIII-XXIII line in FIG. 22.

FIG. 24 is a schematic view for explaining movements of two bending operation wires corresponding to a rotation of a pulley according to the fourth embodiment.

FIG. 25 is a perspective view of a projecting portion according to a modification 4-1 of the fourth embodiment.

FIG. 26 is a perspective view of the projecting portion showing a state in which two bending operation wires are hooked on the projecting portion according to the modification 4-1 of the fourth embodiment.

FIG. 27 is a side view for explaining positions of the two bending operation wires with respect to a rotating member according to a modification 4-2 of the fourth embodiment.

FIG. 28 is a diagram showing an internal structure of an operation portion according to a fifth embodiment.

FIG. 29 is a perspective view of an inside of a housing member of an operation portion according to a sixth embodiment.

FIG. 30 is a partial sectional view taken along XXX-XXX line in FIG. 29.

FIG. 31 is a schematic diagram for explaining movement of two pulleys corresponding to a rotation of a rotating member according to the sixth embodiment.

FIG. 32 is a schematic diagram for explaining movements of the two pulleys corresponding to the rotation of the rotating member according to the sixth embodiment.

FIG. 33 is a perspective view of an inside of a housing member of an operation portion according to a seventh embodiment.

FIG. 34 is a schematic diagram for explaining movements of two pulleys corresponding to a rotation of a rotating member according to the seventh embodiment.

FIG. 35 is a schematic diagram for explaining movements of the two pulleys corresponding to the rotation of the rotating member according to the seventh embodiment.

### DETAILED DESCRIPTION

In general, when an outer diameter dimension of a rotating member in an operation portion is increased in order to improve operability of insertion device, a size of the operation portion incorporating the rotating member also increases.

When the rotating member is a pulley, a bending operation wire is located on a straight line connecting an outer circumferential surface of the pulley and an end portion of a wire sheath. Therefore, when the pulley is increased in size, a frictional force between the end portion of the wire sheath and the bending operation wire increases and an operation amount of force on a lever and the like also increases. The increased frictional force causes rupture of the bending operation wire in the worst case.

Further, since the bending operation wire is located on the straight line connecting the outer circumferential surface of the pulley and the end portion of the wire sheath, when the pulley is increased in size, the operation portion increases in size by a size of a space of the bending operation wire located on the straight line. Accordingly, the size of the operation portion increases by not only a space capacity for housing the pulley but also a space capacity of the bending operation wire located on the straight line. When the operation portion increases in size as a whole, a grasping portion of the operation portion also increases in size. It is difficult for the user to grasp the grasping portion and operability of the operation portion is deteriorated.

According to embodiments explained below, it is possible to provide insertion device and an operation portion unit that do not deteriorate operability and grasping performance of an operation portion even if an outer diameter of a rotating member such as a pulley is increased.

A plurality of endoscopes as examples of insertion device are explained below. Note that, in the following explanation, it should be noted that drawings based on the respective embodiments are schematic and relations between thicknesses and widths of respective portions, ratios of the thicknesses of the respective portions, and the like are different from real ones. Portions, relations and ratios of dimensions of which are different, are sometimes included among the drawings.

### First Embodiment

### (Configuration of an Endoscope)

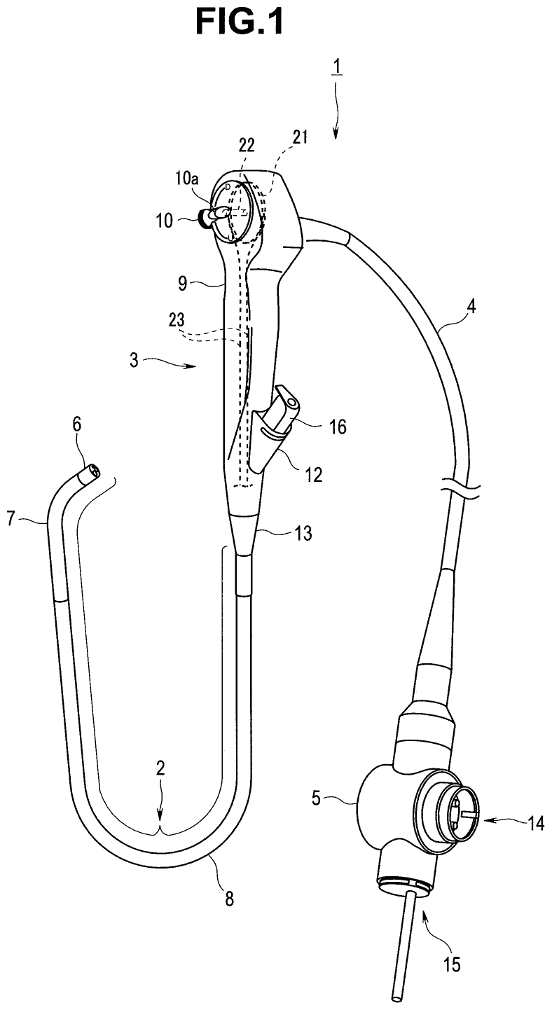

FIG. 1 is an exterior view of an endoscope in the present embodiment. As shown in FIG. 1, an electronic endoscope (hereinafter simply referred to as endoscope) 1 in the present embodiment illustrates a bronchus endoscope. Note that the endoscope 1 is not limited to the bronchus endoscope and may be a so-called upper endoscope introduced from an oral cavity and inserted into a stomach, a duodenum, and the like, a so-called lower endoscope introduced from an anus and inserted into a large intestine, or the like.

The endoscope 1 shown in FIG. 1 is configured mainly by an elongated tubular insertion portion 2, an operation portion 3, a universal cord 4, an endoscope connector 5, and the like. The insertion portion 2 is inserted into a subject from a distal end side in a longitudinal axis direction of the insertion portion 2. The operation portion 3 is provided on a proximal end side of the insertion portion 2. The universal cord 4 extends from the operation portion 3. The endoscope connector 5 is disposed at a distal end of the universal cord 4.

The insertion portion 2 includes a distal end portion 6, a bending portion 7, and a flexible tube portion 8 in order from the distal end side. The distal end portion 6 includes, in a distal end surface, an observation window, an illumination window, and a treatment instrument channel opening portion (all of which are not shown). The insertion portion 2 is formed by consecutively connecting the distal end portion 6, the bending portion 7, and the flexible tube portion 8. The flexible tube portion 8 is a tubular member having flexibility.

The operation portion 3 includes a grasping portion 9, a bending operation lever 10, a treatment instrument insertion portion 12, and a bend preventing portion 13.

The endoscope connector 5 includes an electric connector portion 14 and a light source connector portion 15. Note that the electric connector portion 14 and the light source connector portion 15 are sometimes integrated depending on a model of the endoscope 1.

An image pickup unit and an illumination optical system are incorporated in the distal end portion 6 of the insertion portion 2. The image pickup unit includes a CCD image sensor or a CMOS image sensor, which is an image pickup apparatus. The illumination optical system irradiates a subject with illumination light transmitted through a light guide.

Note that the endoscope 1 is not limited to the electronic endoscope and may be a fiber scope in which an image guide fiber not including an image pickup unit is disposed in the insertion portion 2.

The bending portion 7 is a functional part configured to be able to actively bend in upward and downward two directions (for example, UP-DOWN) according to rotating operation of the bending operation lever 10 by a doctor.

Note that the bending portion 7 is not limited to the bending portion of this type and may be a bending portion of a type that can bend in four directions including left and right directions in addition to the upward and downward directions (an entire circumferential direction around an axis, for example, UP-DOWN/RIGHT-LEFT according to upward, downward, left, and right operations).

The flexible tube portion 8 is formed with flexibility to be passively capable of bending. Although not shown, a treatment instrument insertion channel, various signal lines, a light guide, and the like are inserted through an inside of the flexible tube portion 8.

The various signal lines extend from the image pickup unit of the distal end portion 6 and is extended to an inside of the universal cord 4 through the operation portion 3. The light guide includes an optical fiber formed by one or a plurality of bundles for guiding illumination light from a light source apparatus and emitting the illumination light from the distal end portion 6. The light guide is inserted through insides of the operation portion 3 and the universal cord 4 from the distal end portion 6.

The grasping portion 9 of the operation portion 3 is a portion that the user grasps with a hand when using the endoscope 1. The bending operation lever 10 is an operation member operated when bending operation for the bending portion 7 is performed.

The treatment instrument insertion portion 12 includes a treatment instrument insertion opening. A forceps plug 16 may be disposed in the treatment instrument insertion portion 12. The forceps plug 16 is a lid member for opening and closing the treatment instrument insertion opening and is detachably attachable to the treatment instrument insertion opening (replaceable).

The treatment instrument insertion opening is an opening for inserting various treatment instruments (not shown). The treatment instrument insertion opening communicates with the treatment instrument insertion channel via a branching member on an inside of the operation portion 3. The treatment instrument insertion channel is provided in the insertion portion 2.

The bend preventing portion 13 is provided on a distal end side of the operation portion 3. The bend preventing portion 13 is connected to the flexible tube portion 8 to cover a proximal end of the flexible tube portion 8.

The universal cord 4 is a composite cable through which the various signal lines and the light guide are inserted.

A signal cable for connecting the electric connector portion 14 of the endoscope connector 5 and a video processor (not shown) of external equipment is connected to the electric connector portion 14 of the endoscope connector 5. A light guide bundle and an electric cable (not shown) for connecting the light source connector portion 15 and a light source apparatus, which is external equipment, are connected to the light source connector portion 15. Note that the video processor and the light source apparatus are sometimes an apparatus obtained by integrating the video processor and the light source apparatus.

A pulley 21, which is a rotating member, is provided in the operation portion 3 as indicated by a dotted line in FIG. 1. The bending operation lever 10 includes an arm portion 10a. The bending operation lever 10 is capable of rotating around an axis of a shaft member 22 fixed to one end of the arm portion 10a. A center of the pulley 21 having a disk shape is connected and fixed to the shaft member 22. Accordingly, the pulley 21 rotates according to a rotation of the bending operation lever 10.

Each of one end of the two bending operation wires 23 can be fixed to the pulley 21. Each of the other end of the two bending operation wires 23 is connected to the distal portion. The respective bending operation wires 23 are traction members. The respective bending operation wires 23 are traction members that pull a distal end side portion of the insertion portion 2. The two bending operation wires 23 are inserted into the insertion portion 2.

### (Configuration of the Operation Portion)

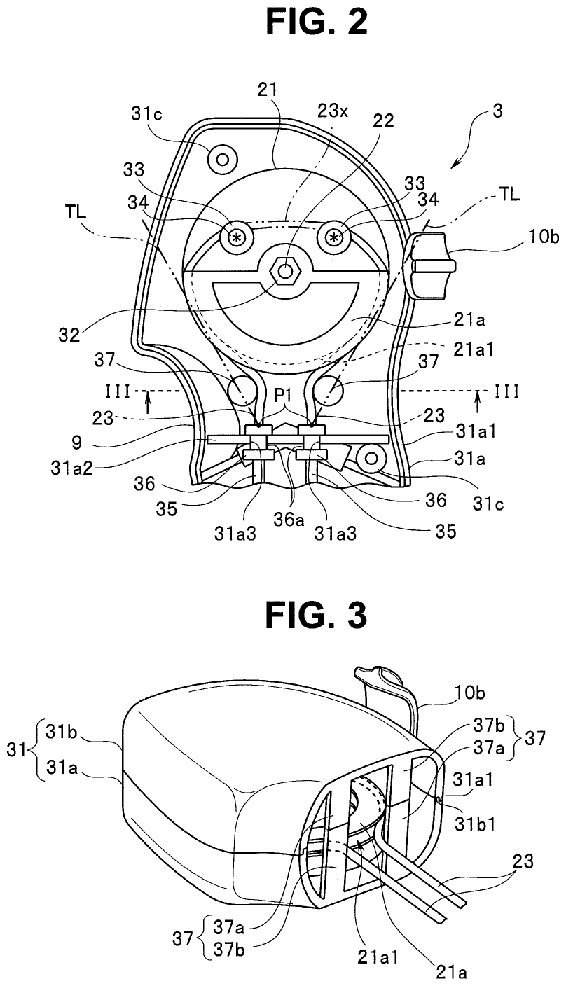

FIG. 2 is a diagram showing an internal structure of the operation portion 3. FIG. 3 is a perspective view of the operation portion showing a cross section of the operation portion taken along line in FIG. 2.

The operation portion 3 includes a housing 31. The pulley 21 is disposed in the housing 31. The housing 31 is configured from two housing members 31a and 31b. Each of the two housing members 31a and 31b is, a molded member having a shell shape made of, for example, resin. A space for housing the pulley 21 is formed on an inside of the housing 31 by engaging respective step portions 31a1 and 31b1 of the housing members 31a and 31b.

A plurality of bosses 31c are provided in the housing member 31a. Although not shown, pin members that enter holes of the respective bosses 31c are provided in the housing member 31b. The respective pin members fit in the holes of the bosses 31c when the respective step portions 31a1 and 31b1 of the two housing members 31a and 31b are engaged. Consequently, the housing 31 having strength is formed. Note that the two housing members 31a and 31b may be structured to be fixed by a screw. In that case, for example, a hole through which the screw is inserted is provided in one of the two housing members 31a and 31b.

FIG. 2 shows disposition of various components disposed in the housing member 31a. On the inside of the operation portion 3, the shaft member 22 is fixed to bearing portions (not shown) provided in the housing members 31a and 31b to be able to rotate together with the pulley 21 around an axis of the shaft member 22 itself.

A shaft of the bending operation lever 10 has a hexagonal outer diameter shape with a hole opened in a center of the shaft. A hexagonal hole is formed in the center of the pulley 21 as well. The bending operation lever 10 is fixed to the pulley 21 by pushing the shaft of the bending operation lever 10 into the hole and screwing a screw into the hole of the bending operation lever 10. Accordingly, when the shaft member 22 rotates around the axis, the pulley 21 also rotates around the axis of the shaft member 22.

The pulley 21 includes a semicircular portion 21a. The semicircular portion 21a includes a groove 21a1 formed in an outer circumferential portion of a semicircle.

As shown in FIG. 2, each of the two bending operation wires 23 is connected to a part other than the semicircular portion 21a of the pulley 21 by washers 33 and screws 34. Note that nuts for fastening the screws 34 may be present on a rear side of the pulley 21. In a state in which parts of the respective bending operation wires 23 are locked to the groove 21a1 of the semicircular portion 21a, one ends of the respective bending operation wires 23 are fixed to the pulley 21.

As explained above, the pulley 21 is the rotating member that rotates, with an external operation force, around the axis of the shaft member 22 provided in the operation portion 3.

The two bending operation wires 23 are respectively inserted through two sheaths 35. The sheaths 35 are wire sheaths or guide coils through which the bending operation wires 23 are inserted. The two sheaths 35 are inserted in parallel through the operation portion 3 and the flexible tube portion 8 along a central axis of the flexible tube portion 8. In other words, the respective sheaths 35 are adjacently inserted through the insertion portion 2. The bending operation wires 23 are inserted through insides of the respective sheaths 35. Longitudinal axes of the sheaths 35 may be parallel to each other. Outside of walls of the sheaths may be parallel to each other. The meaning of “parallel” may include substantially parallel in this disclosure.

One ends of the respective bending operation wires 23 extending from end portions of the sheath 35 are fixed to the pulley 21. The other ends of the respective bending operation wires 23 are fixed to the distal end side portion of the insertion portion 2. More specifically, the other ends of the respective bending operation wires 23 are fixed to predetermined positions of distal end bending pieces among a plurality of bending pieces of the bending portion 7.

Fastening members 36 having a cylindrical shape are fixed to end portions of the respective sheaths 35. The respective fastening members 36 include circumferential grooves 36a in centers.

Note that, here, the two bending operation wires 23 are inserted through the insertion portion 2. However, as indicated by an alternate long and two short dashes line in FIG. 2, a center portion 23x of one bending operation wire 23 may be fixed to the pulley 21. An extending portion on one side of the one bending operation wire 23 may be inserted through one of the two sheaths 35 and an extending portion of the other side of the one bending operation wire 23 may be inserted through the other of the two sheaths 35.

In other words, portions equivalent to the two bending operation wires 23 explained above inserted through the two sheaths 35 may be formed by the one bending operation wire 23. In other words, the one bending operation wire 23 includes two extending portions. In that case, the portions equivalent to the two bending operation wires 23 in FIG. 2 are two extending portions of the one bending operation wire 23 extending from the pulley 21. As shown in FIG. 2, the two extending portions extend from the two sheaths 35. The respective extending portions are parts of the bending operation wires 23 extending in parallel from the two sheaths 35 in the operation portion 3.

The housing member 31a includes a fastening plate portion 31a2 in a shell. Although not shown, the housing member 31b also includes a fastening plate portion in a shell. Two cutout portions 31a3 are formed in the fastening plate portion 31a2.

The two fastening plate portions come into contact when the respective step portions 31a1 and 31b of the two housing members 31a and 31b are engaged. At this time, the circumferential grooves 36a of the respective fastening members 36 engage in the cutout portions 31a3 of the fastening plate portion 31a2.

The circumferential grooves 36a are engaged in the cutout portions 31a3 of the fastening plate portion 31a2, whereby the end portions of the respective sheaths 35 are fixed to the housing 31 in the housing 31.

The two bending operation wires 23 advance and retract in a central axis direction of the bending operation wires 23 according to a movement of the bending operation lever 10. Two guides 37 that guide routes of movements of the two bending operation wires 23 are provided in the housing 31. The two guides 37 guide two extending portions of the two bending operation wires 23 explained above. The two guides 37 are configured to guide the two bending operation wires 23 from the sheaths 35 toward a circumference of the pulley 21.

Each of the two guides 37 has a columnar shape. As shown in FIG. 2, center axes of the respective guides 37 are parallel to a central axis of the shaft member 22. Two projecting portions 37a are provided on an inner side of the shell of the housing member 31a. Similarly, two projecting portions 37b are provided on an inner side of the shell of the housing member 31b.

Each of the four projecting portions 37a and 37b may be a part of the two housing members 31a and 31b or may be configured by another member attached to inner walls of the two housing members 31a and 31b. In other words, the two guides 37 are fixed in the operation portion 3.

Note that, here, length (a projection amount) of the projecting portions 37a and length (a projection amount) of the projecting portions 37b are different such that the operation portion 3 is easily assembled. The length (the projection amount) of the projecting portions 37a is larger than the length (the projection amount) of the projecting portions 37b.

Furthermore, antifriction machining may be applied to surfaces of the respective guides 37. The antifriction machining means, for example, applying fluorine coat to the surfaces of the respective guides 37 or providing tube members made of a fluorine material in parts that are in contact with the bending operation wires 23.

When the respective step portions 31a1 and 31b1 of the two housing members 31a and 31b are engaged, two distal end faces of the two projecting portions 37a and distal end faces of the two projecting portions 37b come into contact and two guides 37 having a columnar shape are formed in the housing 31.

The respective guides 37 are shaft members provided near the pulley 21 to bring the two bending operation wires 23 close to each other around the pulley 21. More specifically, the respective guides 37 are disposed such that, between the fastening members 36 and the semicircular portion 21a, the respective bending operation wires 23 pass the shaft member 22 side with respect to tangential lines TL (indicated by alternate long and two short dashes lines) of the semicircular portion 21a passing a point P1 on one ends of the sheaths 35 (one ends of the fastening members 36).

In other words, the respective guides 37 are disposed on the shaft member 22 side with respect to the tangential lines TL of an outer circumference of the pulley 21 passing a point on one ends of the sheaths 35 in the operation portion 3 and guide the bending operation wires 23 to pass the shaft member 22 side with respect to the tangential lines TL.

As shown in FIG. 2, moving routes (advancing and retracting routes) of the respective bending operation wires 23 are restricted by the respective guides 37 such that, when viewed in a direction parallel to the central axis of the shaft member 22, the respective bending operation wires 23 extending from the fastening members 36 pass the shaft member 22 side with respect to the tangential lines TL. At least a portion of the at least one guides 37 is located on the shaft member 22 side with respect to the tangential line TL of an outer circumference of the pulley 21. The at least one guides 37 is configured to guide the at least one bending operation wire 23 such that at least a portion of a path of the at least one bending operation wire 23 passes on the shaft member 22 side of the tangential line TL.

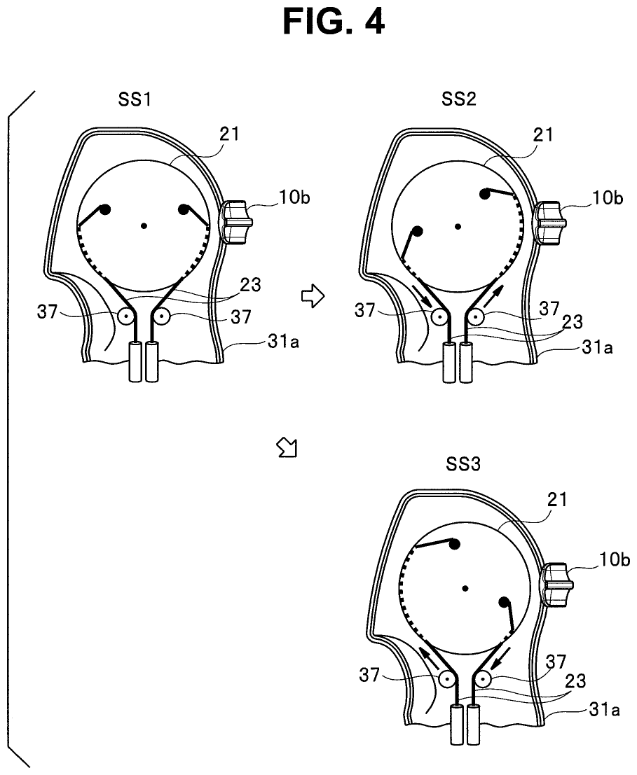

FIG. 4 is a schematic diagram for explaining movements of the two bending operation wires 23 corresponding to a rotation of the pulley 21. As shown in FIG. 4, when the pulley 21 rotates to pull one of the two bending operation wires 23, the two bending operation wires 23 change from a state SS1 to a state SS2 or a state SS3.

With such a configuration, on an inner side of the grasping portion 9 of the housing 31, the respective bending operation wires 23 pass the shaft member 22 side with respect to the tangential lines TL described above. Therefore, even if an outer diameter of the pulley 21 increases, a portion of the grasping portion 9 in the operation portion 3 can be thinned.

Accordingly, even if the outer diameter of the pulley 21 increases, a surgeon can easily grasp the grasping portion 9. Therefore, deterioration in operability of the operation portion 3 can be prevented.

When bending operation is performed many times and the pulley 21 advances and retracts many times, it is likely that a slacked bending operation wire 23 of the two bending operation wires 23 slips off a groove 21c of the pulley 21. However, the two guides 37 are formed to connect inner walls in the housing 31. Therefore, since movement in an axial direction of the guides 37 of the respective bending operation wires 23 is restricted, the slacked bending operation wire 23 less easily slips off the groove 21c of the pulley 21.

As explained above, according to the embodiment explained above, it is possible to provide insertion device that does not deteriorate operability and grasping performance of the operation portion 3 even if an outer diameter of a rotating member such as the pulley 21 is increased. According to the embodiment explained above, it is also possible to achieve an effect of reducing friction between the wires 23 and the sheaths 35. At least one or more of the guides 37 and the sheath 35 is configured to guide the wires 23 such that friction generated between the wires 23 and the sheaths 35 is reduced.

Subsequently, modifications of the embodiment explained above are explained.

In the respective modifications explained below, the same components as the components in the embodiment are denoted by the same numbers and the like and explanation of the components is omitted. Components different from the components in the embodiment are explained.

### Modification 1-1

In the embodiment explained above, the two guides 37 that guide the routes of the movements of the two bending operation wires 23 are the two columnar portions formed by parts of the two housing members 31a and 31b. However, in a modification 1-1, each of the two guides 37 are a pulley or a bearing provided at a shaft member.

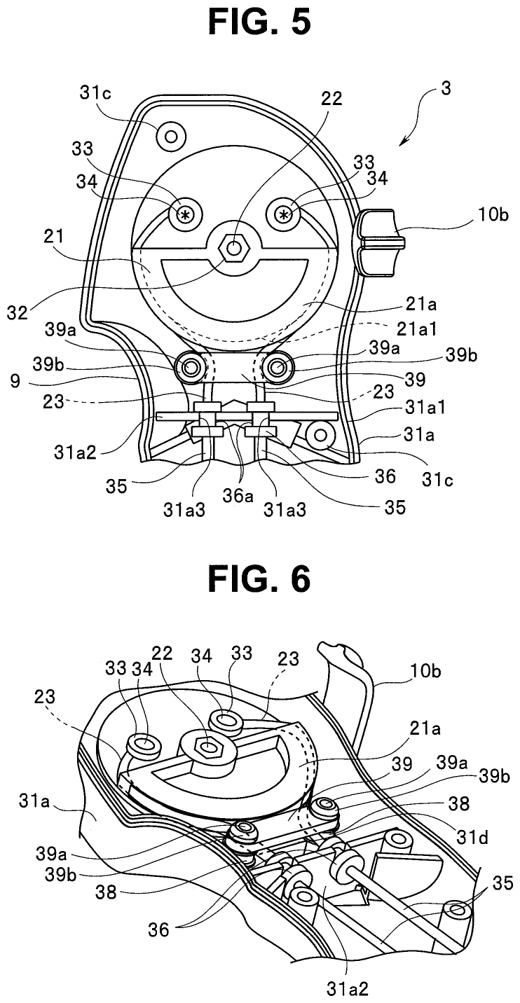

FIG. 5 is a diagram showing an internal structure of the operation portion 3 according to the modification 1-1. FIG. 6 is a perspective view of an inside of the housing member 31a of the operation portion 3 according to the modification 1-1.

As shown in FIG. 6, two pulleys 38 are rotatably fixed between a plane portion of a rib 31d and a slip-off preventing plate 39 formed in the housing member 31a. The respective pulleys 38 are provided at shaft members 39a provided in the housing member 31a to be capable of rotating around axes of the shaft members 39a.

The slip-off preventing plate 39 is fixed to two shaft members 39a by two nuts 39b. Note that the shaft members 39a may be configured by screws and screwed and fixed in holes provided in the rib 31d. The two bending operation wires 23 are disposed to pass between the two pulleys 38 and between the rib 31d and the slip-off preventing plate 39.

Each of the two pulleys 38 configures a guide member corresponding to the guide 37 explained above. In other words, the respective pulleys 38 configure guide members provided at the shaft members 39a and capable of rotating around the axes of the shaft members 39a. By using the two pulleys 38, friction that occurs in the guide member and the bending operation wires 23 can be eliminated and an increase in an operation amount of force can be prevented for the user.

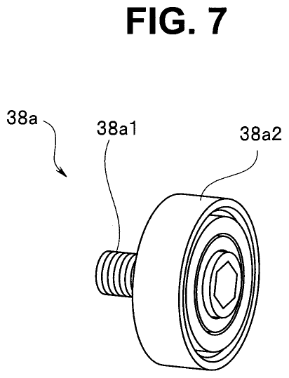

Note that bearings may be used instead of the pulleys 38. FIG. 7 is a perspective view of a bearing 38a. In the bearing 38a, a cylindrical rotating portion 38a2 is provided at one end of a shaft member 38a1 so as to rotate around an axis of the shaft member 38a1. Accordingly, each of the two pulleys 38 shown in FIGS. 5 and 6 may be the bearing 38a.

Furthermore, a guide unit 39A obtained by integrally molding the slip-off preventing plate 39 explained above, the rib 31d, and the two guides 37 may be used without using the pulleys 38 or the bearing 38a.

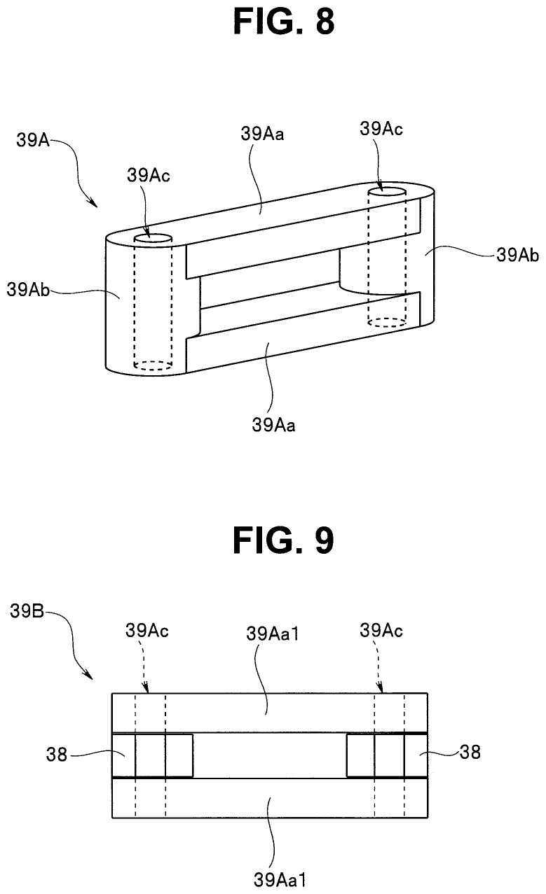

FIG. 8 is a perspective view of the guide unit 39A. The guide unit 39A is made of resin and can be manufactured by integral molding. The guide unit 39A includes two plate-like portions 39Aa corresponding to the slip-off preventing plate 39 explained above and the rib 31d and two columnar portions 39Ab corresponding to the two pulleys 38.

In the guide unit 39A, the two bending operation wires 23 can pass between the two columnar portions 39Ab and between the two plate-like portions 39Aa. Two screw holes 39Ac are provided in two positions corresponding to the two columnar portions 39Ab. The guide unit 39A is fixed to the housing member 31a by inserting screws through the two screw holes 39Ac.

The two columnar portions 39Ab of the guide unit 39A configure a guide member that restricts movements of the two bending operation wires 23. Since the guide unit 39A is an integrally molded member, the guide unit 39A leads to a reduction in the number of components and a reduction in assembly cost.

Note that the guide unit 39A may not be the integrally molded member and may be formed by coupling two or more members.

Furthermore, the columnar portions 39Ab of the guide unit 39A may be replaced with the pulleys 38. FIG. 9 is a side view of a guide unit 39B in which the columnar portions 39Ab are replaced with the pulleys 38.

The two pulleys 38 are disposed to be sandwiched between two plate members 39Aa1. The two pulleys 38 are capable of rotating around center axes of screws (not shown) passing through the screw holes 39Ac. In the guide unit 39B shown in FIG. 9 as well, since the two pulleys 38 are used, friction in the guide can be eliminated and an increase in an operation amount of force can be prevented for the user.

### Modification 1-2

In the modification 1-1, the guide unit 39A is provided to be fixed to the rib of one housing member of the housing 31. However, in a modification 1-2, the two pulleys 38 are fixed to two bosses formed in one of the two housing members 31a and 31b.

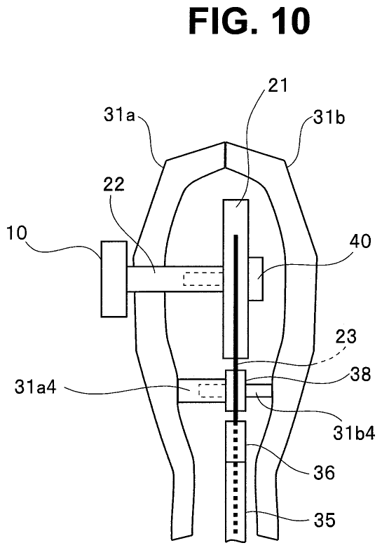

FIG. 10 is a schematic sectional view of the operation portion 3 according to the modification 1-2. FIG. 10 shows a cross section passing the axis of the shaft member 22 and parallel to a longitudinal axis of the operation portion 3.

The pulley 21 is fixed to a shaft of the shaft member 22 by a screw 40. Two bosses 31a4 are formed in predetermined positions on an inner side of the housing member 31a. The two pulleys 38 are rotatably provided at end portions of the two bosses 31a4.

Two pins 31b4 are formed on an inner side of the housing member 31b. The two pins 31b4 are formed on the inner side of the housing member 31b such that the two pins 31b4 are pressed into distal end portions of the two bosses 31a4 when the respective step portions 31a1 and 31b1 of the two housing members 31a and 31b are engaged. When the two pins 31b4 are pressed into the distal end portions of the two bosses 31a4, the two pulleys 38 are rotatably supported in the housing 31.

According to the modification 1-2, since the two pulleys 38 are used, friction in the guide can be eliminated and an increase in an operation amount of force can be prevented for the user. Since the bosses and the pins provided in the housing members 31a and 31b are used, it is possible to achieve a reduction in the number of components and a reduction in assembly cost.

### Modification 1-3

In the modification 1-2, the two bosses formed in one of the two housing members 31a and 31b and the two pins formed in the other are used. However, in a modification 1-3, the two pulleys 38 are fixed using two bosses formed in one of two housing members.

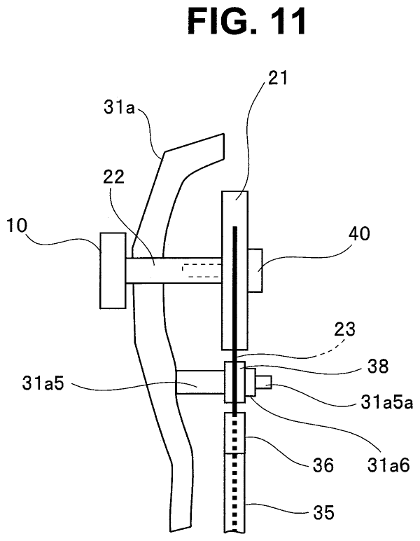

FIG. 11 is a schematic sectional view of the operation portion 3 according to the modification 1-3. FIG. 11 shows a cross section passing the axis of the shaft member 22 and parallel to the longitudinal axis of the operation portion 3.

A boss 31a5 formed in the housing member 31a includes a pin 31a5a at a distal end. The pulley 38 is provided at the distal end of the boss 31a5. A ring-like pressing member 31a6 is pressed into the pin 31a5a, whereby the pulley 38 is fixed to the boss 31a5.

According to the modification 1-3, the same effects as the effects in the modification 1-2 are achieved.

### Modification 1-4

In the embodiment explained above, the two guides 37 are disposed in the housing 31 such that the center axes of the two guides 37 are parallel. However, in a modification 1-4, the two guides 37 are disposed in the housing 31 such that two center axes of the two guides 37 are coaxial.

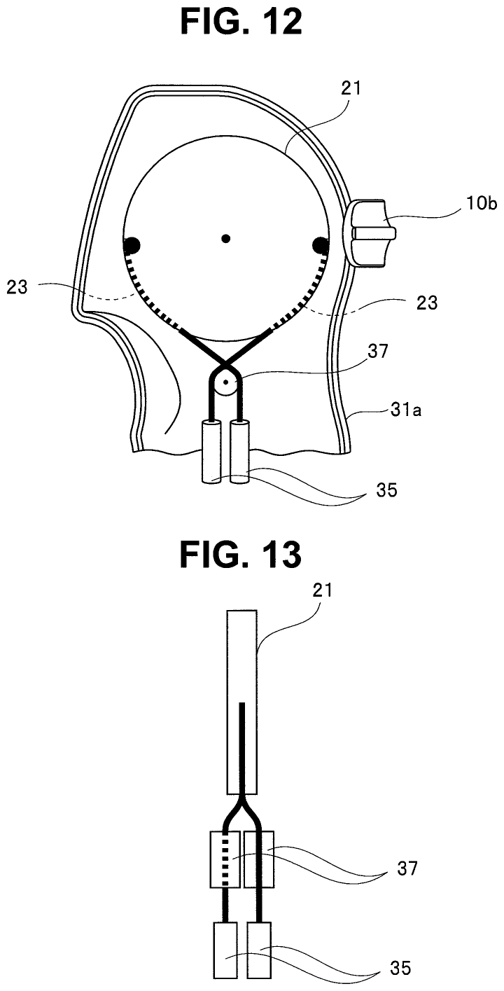

FIG. 12 is a schematic diagram showing an internal structure of the operation portion 3 according to the modification 1-4. FIG. 13 is a schematic side view of the operation portion 3 viewed in a direction parallel to the axis of the shaft member 22 and orthogonal to the longitudinal axis of the operation portion 3.

The two guides 37 have a columnar shape. As shown in FIGS. 12 and 13, the two guides 37 are disposed in the housing 31 such that two center axes of the two guides 37 are coaxial.

One ends of the two bending operation wires 23 are fixed to an outer circumferential groove of the pulley 21 having a disk shape. As shown in FIG. 13, the two guides 37 are disposed in positions deviating from a plane of the pulley 21. Therefore, the two bending operation wires 23 deviate from the plane of the pulley 21 and extend toward the two guides 37. Two center axes of the two guides 37 are coaxial.

With a configuration in the modification 1-4, since the two guides 37 are coaxially disposed, the grasping portion 9 can be further thinned.

Note that, in the modification 1-4 as well, the respective guides 37 may be pulleys or bearings. In this case, two pulleys or two bearings, which are the two guides 37, are capable of rotating around the same axis.

### Second Embodiment

In the first embodiment, the two guides 37 are provided in the operation portion 3 with respect to the two bending operation wires. However, in a second embodiment, one guide is provided in the operation portion 3 with respect to the two bending operation wires.

A configuration of an endoscope in the second embodiment is substantially the same as the configuration of the endoscope 1 in the first embodiment. The same components as the components in the first embodiment are denoted by the same reference numbers and the like and explanation of the components is omitted. Components different from the components in the first embodiment are explained.

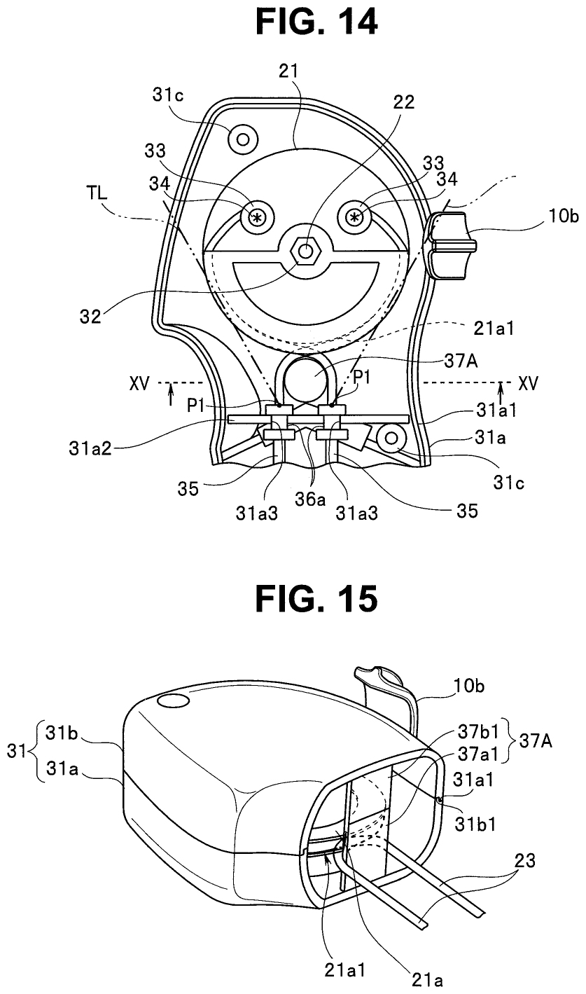

FIG. 14 is a diagram showing an internal structure of the operation portion 3. FIG. 15 is a perspective view of the operation portion showing a cross section of the operation portion taken along XV-XV line in FIG. 14.

As shown in FIGS. 14 and 15, one guide 37A is provided in the operation portion 3. The guide 37A has a columnar shape. As shown in FIG. 14, an axis of the guide 37A is parallel to the central axis of the shaft member 22. A projecting portion 37a1 is provided on the inner side of the shell of the housing member 31a. Similarly, a projecting portion 37b1 is provided on the inner side of the shell of the housing member 31b.

Two projecting portions 37a1 and 37b1 may be respectively parts of the two housing members 31a and 31b or may be configured by other members attached to the inner walls of the two housing members 31a and 31b.

Note that, here, length (a projection amount) of the projecting portion 37a1 and length (a projection amount) of the projecting portion 37b1 are different such that the operation portion 3 is easily assembled. The length (the projection amount) of the projecting portion 37a1 is larger than the length (the projection amount) of the projecting portion 37b1.

Furthermore, antifriction machining may be applied to surfaces of the respective guides 37. The antifriction machining means, for example, applying fluorine coat to the surfaces of the respective guides 37A or providing tube members made of a fluorine material in parts that are in contact with the bending operation wires 23.

As shown in FIG. 14, the two bending operation wires 23 are hooked on the guide 37A to cross in a periphery of the pulley 21 when viewed in the direction parallel to the central axis of the shaft member 22. In other words, one guide 37A guides the two bending operation wires 23.

In the present embodiment as well, as shown in FIG. 14, moving routes (advancing and retracting routes) of the respective bending operation wires 23 are restricted by the guide 37A such that the respective bending operation wires 23 extending from the fastening members 36 pass the shaft member 22 side with respect to the tangential lines TL when viewed in the direction parallel to the central axis of the shaft member 22. At least a portion of the at least one guide member 37 is located on the shaft member 22 side with respect to the tangential line TL of an outer circumference of the pulley 21. The at least one guide member 37 is configured to guide the at least one bending operation wire 23 such that at least a portion of a path of the at least one bending operation wire 23 passes on the shaft member 22 side of the tangential line TL.

With such a configuration, on the inner side of the grasping portion 9 of the housing 31, the respective bending operation wires 23 pass the shaft member 22 side with respect to the tangential lines TL described above. Therefore, even if the outer diameter of the pulley 21 increases, a portion of the grasping portion 9 can be thinned in the operation portion 3.

Accordingly, even if the outer diameter of the pulley 21 increases, a surgeon can easily grasp the grasping portion 9. Therefore, deterioration in operability of the operation portion 3 can be prevented.

Further, since only one guide 37A is provided, effects of simplification of structure of the operation portion 3, a reduction in material expenses, and space saving on the inside of the operation portion 3 are achieved.

As explained above, according to the embodiment explained above, it is possible to provide insertion device that does not deteriorate operability and grasping performance of the operation portion 3 even if an outer diameter of a rotating member such as the pulley 21 is increased.

Subsequently, modifications of the second embodiment explained above are explained.

In the respective modifications explained below, the same components as the components in the first and second embodiments are denoted by the same numbers and the like and explanation of the components is omitted. Components different from the components in the first and embodiments are explained.

### Modification 2-1

In the second embodiment explained above, the guide 37A that guides the routes of the movements of the two bending operation wires 23 are the two columnar portions formed by parts of the two housing members 31a and 31b. However, in a modification 2-1, a guide 37Aa is a pulley held between two plate-like members.

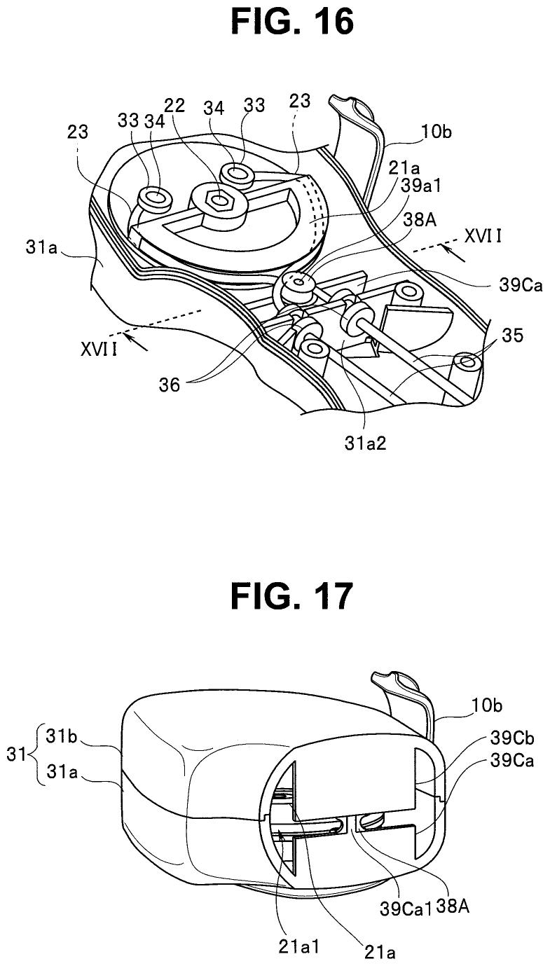

FIG. 16 is a perspective view of an inside of the housing member 31a of the operation portion 3 according to the modification 2-1. FIG. 17 is a perspective view of the operation portion showing a cross section of the operation portion taken along XVII-XVII line in FIG. 16.

As shown in FIGS. 16 and 17, the housing member 31a includes a slip-off preventing plate 39Ca formed on an inner side and the housing member 31b includes a slip-off preventing plate 39Cb formed on an inner side. The slip-off preventing plate 39Ca includes a shaft portion 39Ca1 at a distal end. A pulley 38A is provided at the shaft portion 39Ca1 to be capable of rotating around an axis of the shaft portion 39Ca1.

When the respective step portions 31a1 and 31b1 of the two housing members 31a and 31b are engaged, a distal end of the shaft portion 39Ca1 comes into contact with the slip-off preventing plate 39b and the pulley 38A is held in the operation portion 3 not to slip off the shaft portion 39Ca1.

In the modification as well, the two bending operation wires 23 are hooked on the pulley 38A to cross in the vicinity of the pulley 21 when viewed in the direction parallel to the central axis of the shaft member 22. In other words, one pulley 38A guides the two bending operation wires 23.

The pulley 38A configures a guide corresponding to the guide 37A explained above. By using the pulley 38A, friction in the guide can be eliminated and an increase in an operation amount of force can be prevented for the user.

Note that the bearing shown in FIG. 6 may be used instead of the pulley 38A.

### Third Embodiment

In the first and second embodiments, the guide 37 and the like are provided between the end portions of the sheaths 35 and the pulley 21. However, in a third embodiment, two guides are provided on an opposite side of the end portions of the sheaths 35 with respect to the central axis of the pulley 21.

A configuration of an endoscope in the third embodiment is substantially the same as the configuration of the endoscope 1 in the first embodiment. Therefore, the same components as the components in the first embodiment are denoted by the same numbers and the like and explanation of the components is omitted. Components different from the components in the first embodiment are explained.

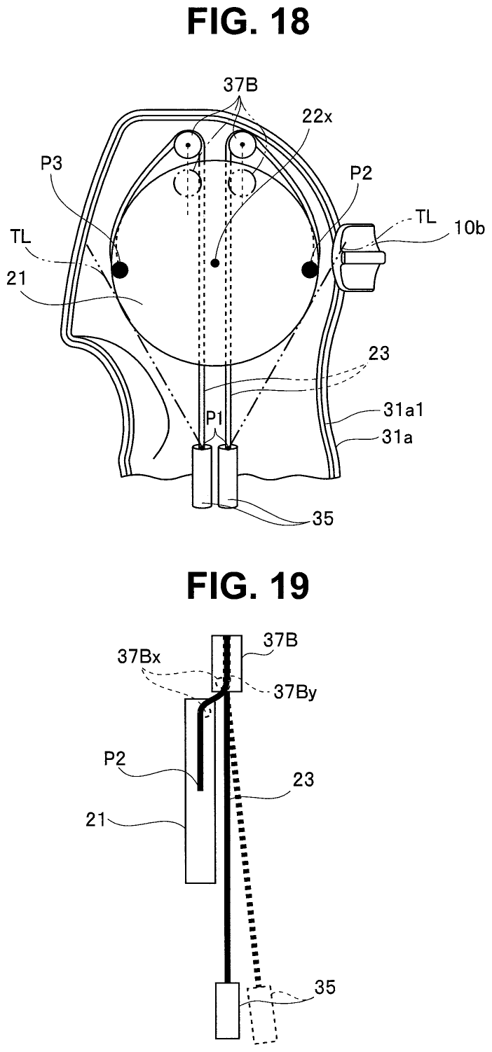

FIG. 18 is a schematic diagram showing an internal structure of the operation portion 3 showing disposition of a pulley and two guides according to the third embodiment. FIG. 18 shows only the pulley 21, two guides 37B, and two sheaths 35. FIG. 19 is a schematic side view of the operation portion 3 showing the disposition of the pulley and the two guides in the operation portion 3 according to the third embodiment.

The respective guides 37B may be the columnar members provided in the housing members 31a and 31b explained in the first embodiment or may be pulleys, bearings, or the like provided in the housing members 31a and 31b. As shown in FIG. 18, the two guides 37B are provided on an opposite side of the insertion portion 2 in a longitudinal direction with respect to a central axis 22x of the shaft member 22.

As shown in FIGS. 18 and 19, the two bending operation wires 23 are drawn out in a direction parallel to the two sheaths 35 from end portions of the two sheaths 35. The two bending operation wires 23 are hooked on the two guides 37B provided on an opposite side of the end portions of the two sheaths 35 (an upper side in FIG. 18) with respect to a central axis of the pulley 21.

After being hooked on the two guides 37B, the two bending operation wires 23 extend in opposite directions each other. Each of a distal end of the two bending operation wires 23 is fixed to a circumferential groove 21a of the pulley 21.

The pulley 21 is capable of rotating around the central axis 22x shown in FIG. 18. Distal ends of the two bending operation wires 23 are respectively fixed to the circumferential groove 21a of the pulley 21 in fixed positions indicated by two points P2 and P3.

Note that, as shown in FIG. 18, central axes of the two guides 37B are located further on an outer side than the outer circumference of the pulley 21 when viewed in the direction parallel to the central axis of the shaft member 22. However, as indicated by an alternate long and two short dashes line, the center axes of the two guides 37B may be disposed to be located further on an inner side than the outer circumference of the pulley 21. In other words, at least a part of the guides 37B may be located within the outer diameter of the pulley 21 when viewed in the direction parallel to the rotary central axis of the pulley 21.

Furthermore, in order to prevent the respective bending operation wires 23 from slipping off the guides 37B, the operation portion 3 may include a movement restricting portion that restricts movements of the respective bending operation wires 23. In FIG. 19, as indicated by dotted lines, a plurality of movement restricting pins 37Bx that restrict movements of the respective bending operation wires 23 between the pulley 21 and the respective guides 37B may be provided in the operation portion 3.

Circumferential grooves 37By in which the bending operation wires 23 fit may be provided on surfaces of the respective guides 37B. By providing the circumferential grooves 37By on the surfaces of the respective guides 37B, it is possible to prevent the bending operation wires 23 from slipping off the respective guides 37B.

Further, as shown in FIG. 19, as indicated by dotted lines, the end portions of the respective sheaths 35 may be disposed in positions separated from the surface of the pulley 21 such that the bending operation wires 23 separate from the pulley 21.

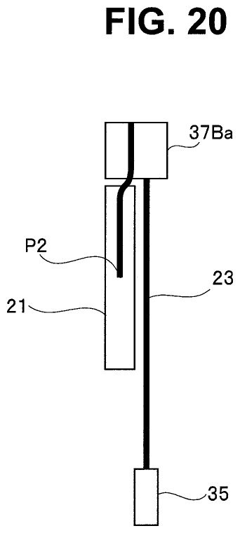

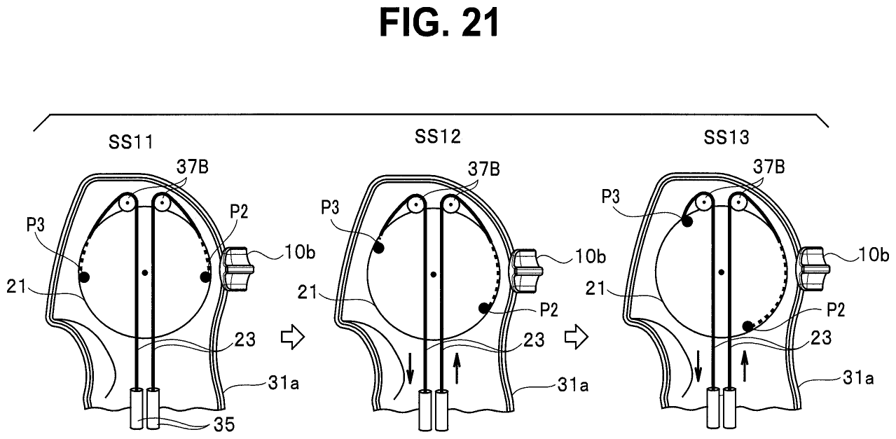

Furthermore, the two guides 37B having a cylindrical shape may be formed longer in the axial direction. In the case of the guide 37B shown in FIG. 19, it is likely that the bending operation wire 23 slips off the guide 37B. Therefore, as shown in FIG. 20, the guide 37B may be formed long in a central axis direction of the guide 37B.

FIG. 20 is a schematic side view of the operation portion 3 showing disposition of the guide 37B long in the central axis direction and the pulley 21 in the operation portion 3.

Note that, in the case of FIG. 20 as well, the movement restricting pin 37Bx shown in FIG. 19 may be provided in the operation portion 3.

FIG. 21 is a schematic diagram for explaining movements of the two bending operation wires 23 corresponding to a rotation of the pulley 21. As shown in FIG. 21, when the pulley 21 rotates to pull one of the two bending operation wires 23, the two bending operation wires 23 changes from a state SS11 to a state SS13 through a state SS12.

With such a configuration, on the inner side of the grasping portion 9 of the housing 31, the respective bending operation wires 23 pass the shaft member 22 side with respect to the tangential lines TL described above. Therefore, even if the outer diameter of the pulley 21 increases, the portion of the grasping portion 9 can be thinned in the operation portion 3.

Accordingly, even if the outer diameter of the pulley 21 increases, a surgeon can easily grasp the grasping portion 9. Therefore, it is possible to prevent deterioration in operability of the operation portion 3.

### Fourth Embodiment

In the first to third embodiments, the end portions of the two bending operation wires 23 are fixed to the pulley 21 provided in the operation portion 3. However, in a fourth embodiment, the end portions of the two bending operation wires 23 are fixed to a rotating member having a rotating range restricting function.

A configuration of an endoscope in the fourth embodiment is substantially the same as the configuration of the endoscope 1 in the first embodiment. The same components as the components in the first embodiment are denoted by the same reference numbers and the like and explanation of the components is omitted. Components different from the components in the first embodiment are explained.

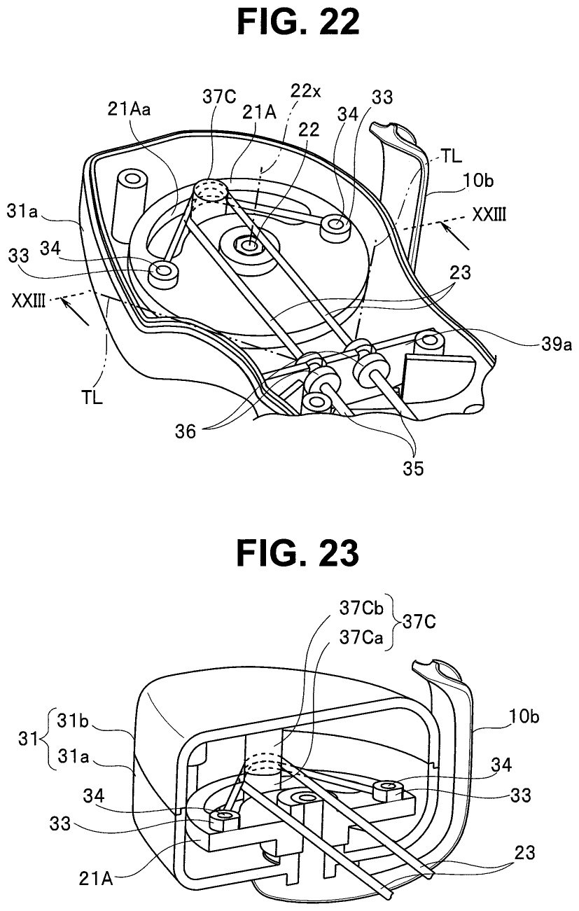

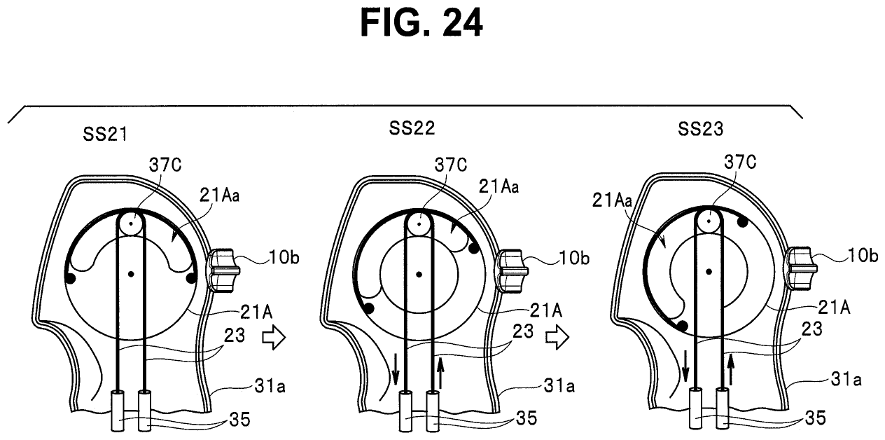

FIG. 22 is a perspective view of an inside of the housing member 31a of the operation portion 3 according to the present embodiment. FIG. 23 is a perspective view of the operation portion showing a cross section of the operation portion taken along XXIII-XXIII line in FIG. 22.

In the operation portion 3, a rotating member 21A capable of rotating around the central axis 22x of the shaft member 22 is provided. The rotating member 21A has a disk shape. The rotating member 21A includes a through-groove 21Aa having an arc shape centering on the central axis 22x of the shaft member 22.

A guide 37C is provided in the housing 31. The guide 37C has a columnar shape. As shown in FIGS. 22 and 23, an axis of the guide 37C is parallel to the central axis 22x of the shaft member 22. The guide 37C is configured from two projecting portions 37Ca and 37Cb. The projecting portion 37Ca is provided on the inner side of the shell of the housing member 31a. Similarly, the projecting portion 37Cb is provided on the inner side of the shell of the housing member 31b. The respective two projecting portions 37Ca and 37Cb may be parts of the two housing members 31a and 31b or may be configured by other members attached to the inner walls of the two housing members 31a and 31b.

The guide 37C is disposed in the arc-shaped through-groove 21Aa. The guide 37C comes into contact with both end portions of the through-groove 21Aa, whereby a rotation of the rotating member 21A is restricted.

The two bending operation wires 23 are hooked on the guide 37C to cross each other when viewed in a direction parallel to the central axis 22x of the shaft member 22. The distal ends of the two bending operation wires 23 are fixed to the washers 33 and the screws 34 near an outer circumferential portions of the rotating member 21A. Note that nuts screwing with the screws 34 may be provided on a rear of the rotating member 21A.

As shown in FIGS. 22 and 23, moving routes (advancing and retracting routes) of the respective bending operation wires 23 are restricted by the respective guides 37C such that, when viewed in the direction parallel to the central axis 22x of the shaft member 22, the respective bending operation wires 23 extending from the fastening members 36 pass the shaft member 22 side with respect to the tangential lines TL. The respective guide 37C is configured to guide the at least one bending operation wire 23 such that at least a portion of a path of the at least one bending operation wire 23 passes on the shaft member 22 side of the tangential line TL.

FIG. 24 is a schematic diagram for explaining movements of the two bending operation wires 23 corresponding to a rotation of the rotating member 21A. As shown in FIG. 24, when the rotating member 21A rotates to pull one of the two bending operation wires 23, the two bending operation wires 23 change from a state SS21 to a state SS23 through a state SS22.

With such a configuration, on the inner side of the grasping portion 9 of the housing 31, the respective bending operation wires 23 pass the shaft member 22 side with respect to the tangential lines TL described above. Therefore, even if an outer diameter of the rotating member 21A increases, a portion of the grasping portion 9 can be thinned in the operation portion 3.

Accordingly, even if the outer diameter of the rotating member 21A increases, a surgeon can easily grasp the grasping portion 9. Therefore, deterioration in operability of the operation portion 3 can be prevented.

Note that, in FIGS. 22 and 23, the rotating member 21A has the disk shape. However, the rotating member 21A may have a semicircular shape if the rotating member 21A includes the through-groove 21Aa and a portion that fixes the distal end portions of the two bending operation wires 23.

Subsequently, modifications of the embodiment explained above are explained.

In the respective modifications explained below, the same components as the components in the embodiment are denoted by the same numbers and the like and explanation of the components is omitted. Components different from the components in the embodiment are explained.

### Modification 4-1

In the embodiment explained above, the guide 37C has a simple columnar shape and the two bending operation wires 23 are only hooked around the guide 37C. However, the guide 37C may include two grooves to prevent the two bending operation wires 23 from interfering with each other around the guide 37C.

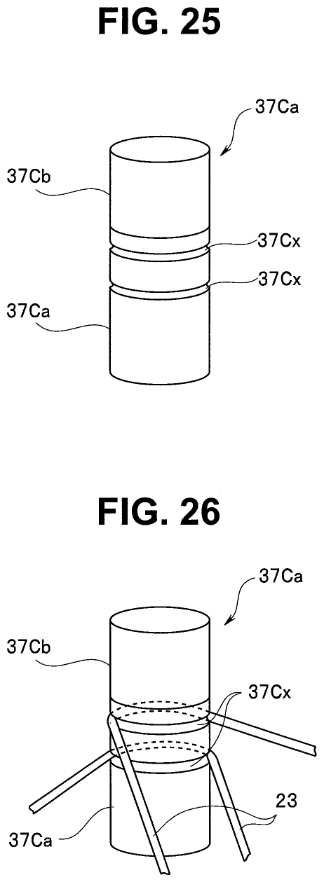

FIG. 25 is a perspective view of the projecting portion 37Ca according to a modification 4-1. FIG. 26 is a perspective view of the projecting portion 37Ca showing a state in which the two bending operation wires 23 are hooked on the projecting portion 37Ca shown in FIG. 25.

Two circumferential grooves 37Cx are formed on an outer circumference of the projecting portion 37Ca. One of the two circumferential grooves 37Cx is for one of the two bending operation wires 23. The other of the two circumferential grooves 37Cx is for the other of the two bending operation wires 23. The respective circumferential grooves 37Cx have a shape in which outer circumferential surfaces of the bending operation wires 23 fit.

The respective bending operation wires 23 enter the circumferential grooves 37Cx of the projecting portion 37Ca. Therefore, the bending operation wires 23 do not interfere around the projecting portion 37Ca when the bending operation wires 23 advance and retract in the axial direction of the bending operation wires 23.

### Modification 4-2

In the embodiment explained above, the two bending operation wires 23 extending from the two fastening members 36 are located in the same plane orthogonal to the central axis 22x of the shaft member 22 of the rotating member 21A. However, the two bending operation wires 23 extending from the two fastening members 36 may not be located in the same plane orthogonal to the central axis of the shaft member 22 of the rotating member 21A.

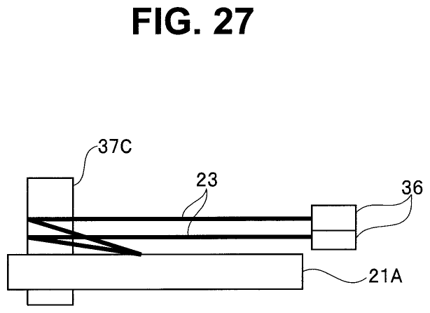

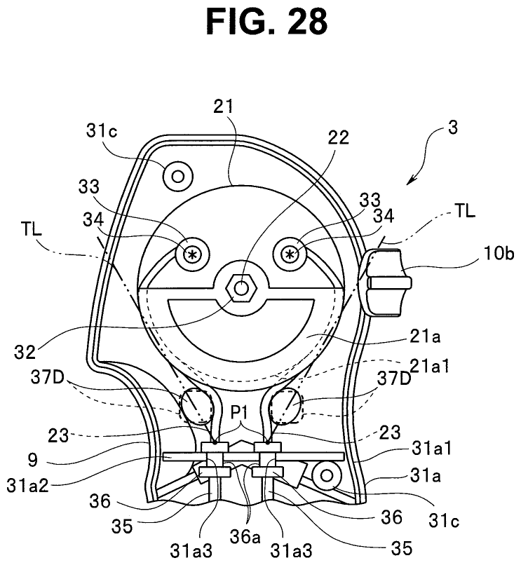

FIG. 27 is a side view for explaining positions of the two bending operation wires 23 with respect to the rotating member 21A according to a modification 4-2. FIG. 27 shows positions of the rotating member 21A and the two bending operation wires 23 viewed in a direction parallel to a surface of the disk-like rotating member 21A.

As shown in FIG. 27, the two fastening members 36 are disposed in different positions in the central axis 22x direction of the shaft member 22. Therefore, it is possible to prevent interference of the two bending operation wires 23.

The modification 4-1 explained above may be applied to the modification 4-2.

### Fifth Embodiment

In the first to fourth embodiments, the example is explained in which the guides have the columnar shape. However, a sectional shape of the respective guides in the direction orthogonal to the axes of the respective guides may be an ellipse, a polygon having corners formed as curved surfaces, or the like.

A configuration of an endoscope in a fifth embodiment is the same as the configuration of the endoscope 1 in the first embodiment. Therefore, the same components as the components in the first embodiment are denoted by the same numbers and the like and explanation of the components is omitted. Components different from the components in the first embodiment are explained.

FIG. 28 is a diagram showing an internal structure of the operation portion 3 according to the present embodiment. In FIG. 28, a sectional shape of guide 37D orthogonal to an axis of the guide 37D is an ellipse.

In the present embodiment as well, it is possible to provide insertion device that does not deteriorate operability and grasping performance of the operation portion 3 even if an outer diameter of a rotating member such as the pulley 21 is increased.

Note that the guides 37D in the embodiment explained above have the elliptical sectional shape. However, the guides 37D may have a square shape having corners formed as curved surfaces as indicated by dotted lines in FIG. 28.

Furthermore, here, the guides 37D have the square shape having the corners formed as the curved surfaces. However, the guides 37D may have another polygonal shape such as a pentagonal shape having corners formed as curved surfaces.

As explained above, according to the embodiment explained above, it is possible to provide insertion device that does not deteriorate operability and grasping performance of the operation portion 3 even if an outer diameter of a rotating member such as the pulley 21 is increased.

### Sixth Embodiment

In the first to fifth embodiments, the two bending operation wires 23 are hooked on one or two guides. However, in the present embodiment, four pulleys are used as guides.

A configuration of an endoscope in a sixth embodiment is substantially the same as the configuration of the endoscope 1 in the first embodiment. The same components as the components in the first embodiment are denoted by the same reference numbers and the like and explanation of the components is omitted. Components different from the components in the first embodiment are explained.

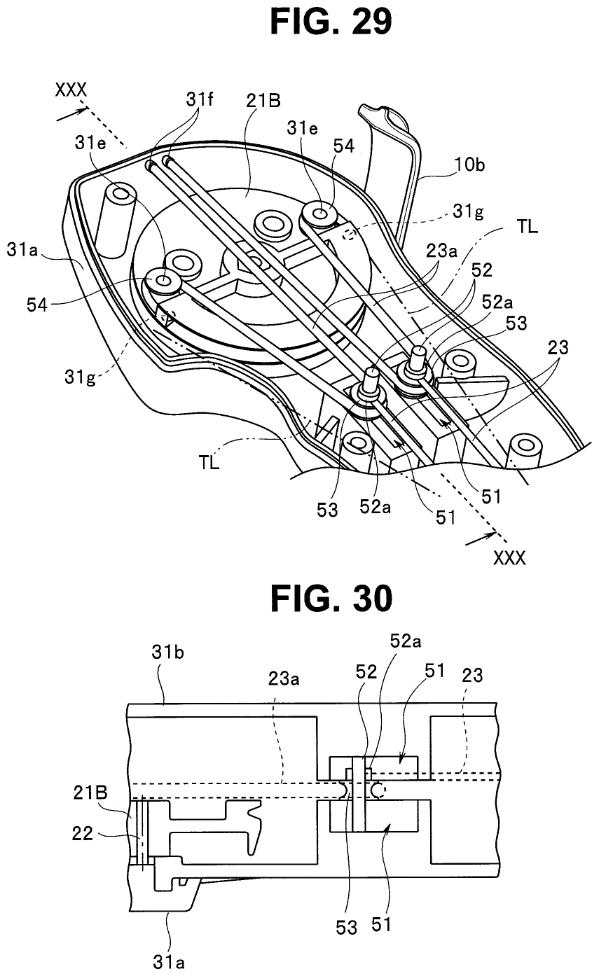

FIG. 29 is a perspective view of an inside of the housing member 31a of the operation portion 3 according to the present embodiment. FIG. 30 is a partial sectional view taken along XXX-XXX line in FIG. 29.

Two grooves 51 are provided in each of the housing members 31a and 31b. The respective grooves 51 are formed, in a longitudinal axis direction of the operation portion 3, in ribs formed in the respective housing members 31a and 31b. The respective grooves 51 have depth in which pin members 52 enter. Pulleys 53 are provided in the pin members 52. The pulleys 53 are located substantially in centers in a longitudinal axis direction of the pin members 52. The pulleys 53 are provided at the pin members 52 to be capable of rotating around axes of the pin members 52.

When the respective step portions 31a1 and 31b1 of the two housing members 31a and 31b are engaged, as shown in FIG. 30, the pin members 52 are disposed in the grooves 51 and the pulleys 53 are disposed between two ribs in which the two grooves 51 are formed. The pin members 52 are capable of moving along the grooves 51. Each of the two pulleys 53 functions as a movable pulley that moves along the groove 51 in the housing 31.

One ends of the bending operation wires 23 are connected to the respective pin members 52 by fastening members 52a. The fastening members 52a are nuts, for example, and screw with male screws formed on outer circumferential surfaces of the pin members 52 to be fixed to the pin members 52. Note that the one ends of the bending operation wires 23 may be fixed to the fastening members 52a by bonding, welding, or the like. Accordingly, the one ends of the bending operation wires 23 are connected to the respective pulleys 53. The respective pulleys 53 are capable of moving in the housing 31 of the operation portion 3.

Two pulleys 54 are fixed to the housing 31 and provided in the housing 31. The respective pulleys 54 are rotatably provided at columnar projecting portions 31e formed in the housing members 31a and 31b. Each of the two pulleys 54 functions as a fixed pulley fixed in the housing 31.

In the housing member 31a, two fixing portions 31f for fixing one ends of two wires 23a are provided. The respective fixing portions 31f are provided on a proximal end side of the housing 31 and may be a rib for fixing the one ends of the respective wires 23a with an adhesive or the like or may be screw holes for fastening the one ends of the respective wires 23a with screws. In other words, the two wires 23a are wires different from the bending operation wires 23, one ends of the wires being fixed to the housing 31.

A disk-like rotating member 21B is provided in the housing 31 to be capable of rotating around the central axis 22x of the shaft member 22. As shown in FIG. 29, the rotating member 21B is connected to one ends of the respective two bending operation wires 23 by the four pulleys 53 and 54 and the two wires 23a.

The respective two wires 23a are hooked on the four pulleys 53 and 54 as shown in FIG. 29. One ends of the two wires 23a are fixed to the fixing portions 31f The respective other ends of the two wires 23a are fixed to fixing portions 31g on the rotating member 21B.

Accordingly, moving routes (advancing and retracting routes) of the respective bending operation wires 23 are restricted by the respective pulleys 53 and 54 such that the respective bending operation wires 23 and the respective wires 23a extending from the fastening members 36 pass the shaft member 22 side with respect to the tangential lines TL when viewed in the direction parallel to the central axis of the shaft member 22. The at least one respective pulley 53 is configured to guide the at least one bending operation wire 23 such that at least a portion of a path of the at least one bending operation wire 23 passes on the shaft member 22 side of the tangential line TL.

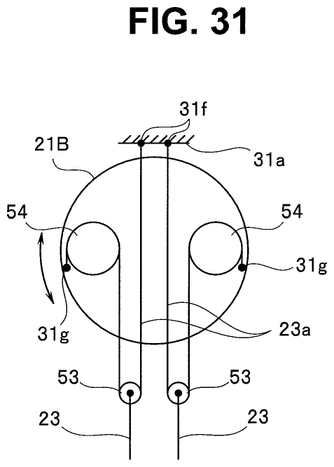

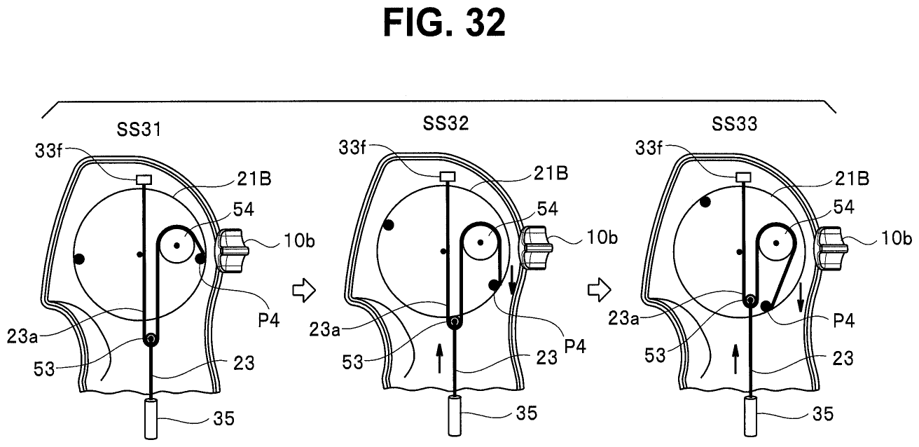

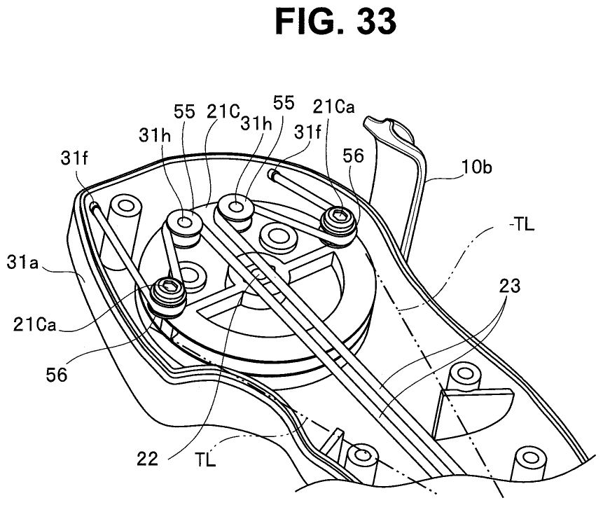

FIGS. 31 and 32 are schematic diagrams for explaining movements of the two pulleys 53 and 54 corresponding to a rotation of the rotating member 21B. FIG. 31 shows positions of the four pulleys 53 and 54 in a state in which both of the two bending operation wires 23 are not pulled.

In FIG. 32, only one pulley 53 and one pulley 54 are shown. In FIG. 32, a point P4 indicates a position of the fixing portion 31g. As shown in FIG. 32, when the rotating member 21B rotates to pull the bending operation wire 23, the point P4 moves and the wire 23a is pulled according to a movement of an outer circumferential portion of the rotating member 21B. As shown in FIG. 32, when the rotating member 21B rotates, a position of the point P4 and a position of the pulley 53, which is a movable pulley, change from a state SS31 to a state SS33 through a state SS32.

In other words, the respective wires 23a are hooked on the pulley 53 and the pulley 54 and move the pulley 53 in a longitudinal axis direction of the bending operation wire 23 according to a rotation of the rotating member 21B.

Accordingly, with such a configuration, since the movable pulley is used, a traction amount of the wire 23 by the pulley 54 is halved but an operation amount of force decreases. In other words, the same effects as effects of a configuration in which the rotating member 21B is substantially increased in size are achieved without increasing the housing 31 of the operation portion 3 in size.

On the inner side of the grasping portion 9 of the housing 31, the respective bending operation wires 23 pass the shaft member 22 side with respect to the tangential lines TL described above. Therefore, even if an outer diameter of the rotating member 21B increases, a portion of the grasping portion 9 can be thinned in the operation portion 3.

Accordingly, even if the outer diameter of the rotating member 21B increases, a surgeon can easily grasp the grasping portion 9. Therefore, deterioration in operability of the operation portion 3 can be prevented.

### Seventh Embodiment

In the sixth embodiment, the two pulleys 53 functioning as the movable pulleys are provided to be capable of moving along the two grooves 51 provided in the housing 31. However, in the present embodiment, the movable pulleys are fixed to the rotating member 21B.

A configuration of an endoscope in a seventh embodiment is substantially the same as the configuration of the endoscope 1 in the first to sixth embodiments. Therefore, the same components as the components in the first to sixth embodiments are denoted by the same numbers and the like and explanation of the components is omitted. Components different from the components in the first to sixth embodiments are explained.

FIG. 33 is a perspective view of an inside of the housing member 31b of the operation portion 3 according to the present embodiment. Two pulleys 55 are fixed to the housing 31. The respective pulleys 55 are rotatably provided at columnar projecting portions 31h formed in the housing member 31b. Each of the two pulleys 55 is a guide member functioning as a fixed pulley fixed in the housing 31.

A disk-like rotating member 21C is provided in the housing 31 to be capable of rotating around the central axis 22x of the shaft member 22. In the rotating member 21C, as shown in FIG. 33, two pulleys 56 are attached near an outer circumferential portion of the rotating member 21C by pin members 21Ca. The respective pulleys 56 are provided to be capable of rotating around axes of the pin members 21Ca. Each of the two pulleys 56 functions as a movable pulley that moves in the housing 31.

The respective two bending operation wires 23 are hooked on the four pulleys 55 and 56 as shown in FIG. 33. One ends of the respective two bending operation wires 23 are fixed to the fixing portions 31f As shown in FIG. 33, the rotating member 21C is connected to the one ends of the respective two bending operation wires 23 using the four pulleys 55 and 56.

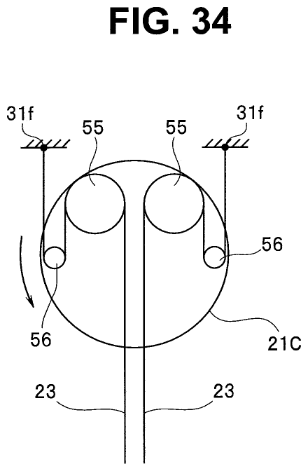

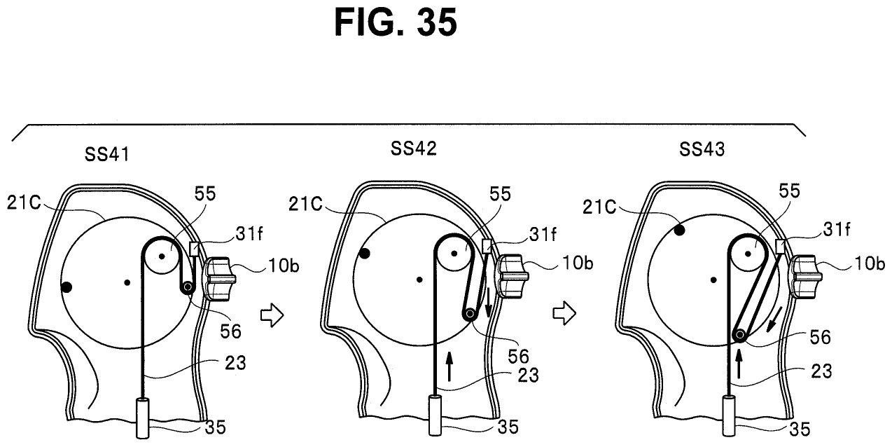

FIGS. 34 and 35 are schematic diagrams for explaining movements of the two pulleys 53 and 54 corresponding to a rotation of the rotating member 21C. FIG. 34 shows positions of the four pulleys 55 and 56 in a state in which both of the two bending operation wires 23 are not pulled.

In FIG. 35, only one pulley 55 and one pulley 56 are shown. As shown in FIG. 35, when the rotating member 21C rotates to pull the bending operation wire 23, the pulley 56 moves along the outer circumferential portion of the rotating member 21C. The bending operation wire 23 is pulled according to the movement of the pulley 56. As shown in FIG. 35, when the rotating member 21C rotates, a position of the pulley 56, which is the movable pulley, becomes a state of SS43 through SS42 from SS 41.

In other words, the respective bending operation wires 23 are hooked on the pulley 55 and the pulley 56. The pulley 56 moves according to the rotation of the rotating member 21C, whereby the respective bending operation wires 23 move in the longitudinal axis direction of the bending operation wires 23.

Accordingly, with such a configuration, since the movable pulley is used, a movement amount of the bending operation wires 23 is larger than a rotating operation amount of the rotating member 21C. Therefore, operability is also high.

On the inner side of the grasping portion 9 of the housing 31, the respective bending operation wires 23 pass the shaft member 22 side with respect to the tangential lines TL described above. Therefore, even if an outer diameter of the rotating member 21C increases, a portion of the grasping portion 9 can be thinned in the operation portion 3.

Accordingly, even if the outer diameter of the rotating member 21C increases, a surgeon can easily grasp the grasping portion 9. Therefore, deterioration in operability of the operation portion 3 can be prevented.

As explained above, according to the respective embodiments and the respective modifications explained above, it is possible to provide insertion device that does not deteriorate operability and grasping performance of an operation portion even if an outer diameter of a rotating member such as a pulley is increased.

Note that, in the respective embodiments explained above, the operation portion 3 is a part of the endoscope 1 functioning as the insertion device. However, the operation portion 3 may be detachable from the endoscope 1 as one operation portion unit. In that case, the operation portion unit is configured such that one end of at least one traction member that pulls a distal end side portion of an insertion portion inserted into a subject can be fixed to the operation portion unit.

The present disclosure is not limited to the respective embodiments explained above. Various changes, alterations, and the like are possible in a range in which the gist of the disclosure invention is not changed.
(Example 1) Insertion equipment comprising:

at least one towing member configured to tow a distal end side portion of an insertion section inserted into a subject;

an operation section provided on a proximal end side of the insertion section;

at least one sheath member inserted through the insertion section, the at least one towing member being inserted through an inside of the at least one sheath member;

a turning member to which one end of the at least one towing member is connected, the turning member turning, with an external operation force, around a first axis of a first shaft member provided in the operation section; and

at least one guide member disposed on the first shaft member side with respect to a tangential line of an outer circumference of the turning member passing a point on one end of the at least one sheath member in the operation section and configured to guide the at least one towing member such that the at least one towing member passes the first shaft member side with respect to the tangential line.
(Example 2) The insertion equipment according to Example 1, wherein the at least one guide member includes a pulley or a bearing capable of turning around an axis of a second shaft member provided in the operation section separately from the first shaft member.
(Example 3) The insertion equipment according to Example 1, wherein

the at least one towing member includes a first extending portion and a second extending portion, and

the at least one guide member includes a first guide that guides the first extending portion and a second guide that guides the second extending portion.
(Example 4) The insertion equipment according to Example 3, wherein

the at least one sheath member includes a first sheath and a second sheath,

the first extending portion extends from the first sheath,

the second extending portion extends from the second sheath, and

the first extending portion and the second extending portion respectively extend in parallel from the first sheath and the second sheath in the operation section.
(Example 5) The insertion equipment according to Example 1, wherein

the at least one guide member includes a first guide and a second guide,

the first guide includes a first pulley or a first bearing,

the second guide includes a second pulley or a second bearing, and

a first turning axis of the first pulley or the first bearing and a first turning axis of the second pulley or the second bearing are coaxial.
(Example 6) The insertion equipment according to Example 1, wherein the at least one guide member is provided on an opposite side of the insertion section with respect to the first shaft member.
(Example 7) The insertion equipment according to Example 1, wherein

the turning member is a pulley, and

at least a part of the at least one guide member is disposed within an outer diameter of the pulley when viewed in a direction parallel to a rotation center axis of the pulley.
(Example 8) The insertion equipment according to Example 1, wherein the first shaft member and the at least one guide member are fixed in an inside of the operation section.
(Example 9) The insertion equipment according to Example 1, comprising:

a first pulley to which one end of the at least one towing member is connected, the first pulley being capable of moving in a housing of the operation section; and

at least one different towing member, one end of which is fixed to the housing, the at least one different towing member being different from the at least one towing member, wherein

another end of the at least one different towing member is fixed to the turning member,

the at least one guide member includes a second pulley fixed to the housing, and

the at least one different towing member is hooked on the first pulley and the second pulley and moves the first pulley in a longitudinal axis direction of the at least one towing member according to a turn of the turning member.
(Example 10) The insertion equipment according to Example 1, comprising a first pulley attached to the turning member, wherein

the at least one guide member includes a second pulley,

one end of the at least one towing member is fixed to a housing of the operation section, and

the at least one towing member is hooked on the first pulley and the second pulley and moves the at least one towing member in a longitudinal axis direction as the first pulley moves according to a turn of the turning member.
(Example 11) An operation section unit that is an operation section unit configured such that one end of at least one towing member that tows a distal end side portion of an insertion section inserted into a subject can be fixed to the operation section unit,

the operation section unit comprising:

a housing;

a turning member provided in the housing and configured to turn, with an external operation force, around a first axis of a first shaft member; and

at least one guide member disposed, in the housing, on the first shaft member side with respect to a tangential line of an outer circumference of the turning member passing a point on one end of the at least one sheath member from which the at least one towing member extends and configured to guide the at least one towing member such that the at least one towing member passes the first shaft member side with respect to the tangential line.

What is claimed is:

1. An insertion device, comprising:
an insertion portion configured to be inserted into a subject;
at least one traction member configured to pull a distal end side portion of the insertion portion;
an operation portion provided on a proximal end side of the insertion portion;
at least one sheath member inserted through the insertion portion,
wherein the at least one traction member is inserted through an inner channel in the at least one sheath member;
a rotating member to which one end of the at least one traction member is connected, the rotating member configured to rotate, with an external operation force, around a first axis of a first shaft member provided in the operation portion; and
at least one guide member disposed in the operation portion,
wherein at least a portion of the at least one guide member is located on a first shaft member side with respect to a tangential line of an outer circumference of the rotating member, the tangential line passing through a point on an end of the at least one sheath member that is located within the operation portion, and
wherein the at least one guide member is configured to guide the at least one traction member such that at least a portion of a path of the at least one traction member passes on the first shaft member side of the tangential line.

2. The insertion device according to claim 1, wherein the at least one guide member comprises a pulley or a bearing configured to rotate around an axis of a second shaft member.

3. The insertion device according to claim 1, wherein the at least one traction member includes a first extending portion and a second extending portion, and
wherein the at least one guide member includes a first guide configured to guide the first extending portion and a second guide configured to guide the second extending portion.

4. The insertion device according to claim 3, wherein:
the at least one sheath member includes a first sheath and a second sheath,
the first extending portion extends from the first sheath,
the second extending portion extends from the second sheath, and
the first extending portion and the second extending portion respectively extend in parallel from the first sheath and the second sheath in the operation portion.

5. The insertion device according to claim 1, wherein:
the at least one guide member includes a first guide and a second guide,
the first guide includes a first pulley or a first bearing,
the second guide includes a second pulley or a second bearing, and
the first pulley or the first bearing has a first rotating axis, the second pulley or
the second bearing has a second rotating axis, and the first rotating axis and the second rotating axis are coaxial.

6. The insertion device according to claim 1, wherein the at least one guide member is provided on an opposite side of the insertion portion with respect to the first shaft member.

7. The insertion device according to claim 1, wherein the rotating member is a pulley, and
wherein at least a part of the at least one guide member is disposed within an outer diameter of the pulley when viewed in a direction parallel to the first axis.

8. The insertion device according to claim 1, wherein the first shaft member and the at least one guide member are fixed to an inner surface of the operation portion.

9. The insertion device according to claim 1, further comprising:
a first pulley to which one end of the at least one traction member is connected, the first pulley being configured to move in a housing of the operation portion; and
a second traction member,
wherein a first end of the second traction member is fixed to the housing and a second end of the second traction member is fixed to the rotating member,
wherein the at least one guide member comprises a second pulley fixed to the housing, and
wherein the second traction member is hooked on the first pulley and the second pulley and configured to move the first pulley in a longitudinal axis direction of the at least one traction member in response to a rotation of the rotating member.

10. The insertion device according to claim 1, further comprising a first pulley attached to the rotating member,
wherein the at least one guide member comprises a second pulley,
wherein one end of the at least one traction member is fixed to a housing of the operation portion, and
wherein the at least one traction member is hooked on the first pulley and the second pulley and configured to move the at least one traction member in a longitudinal axis direction as the first pulley moves in response to a rotation of the rotating member.

11. An operation portion unit configured to fix to one end of at least one traction member configured to pull a distal end side portion of an insertion portion that is configured to be inserted into a subject portion, the operation portion unit comprising:
a housing;
a rotating member provided in the housing and configured to rotate around a first axis of a first shaft member by an external operation force; and
at least one guide member disposed in the housing,
wherein at least a portion of the at least one guide member is located on a first shaft member side with respect to a tangential line of an outer circumference of the rotating member, the tangential line passing through a point on an end of the at least one sheath member, the at least one traction member extends from the at least one sheath member, and
wherein the at least one guide member is configured to guide the at least one traction member such that at least a portion of a path of the at least one traction member passes on the first shaft member side of the tangential line.

12. An insertion device, comprising:
an insertion portion having a distal portion;
a first wire inserted into the insertion portion, the first wire having a first end and a second end, the first end connected to the distal portion;
an operation portion configured to pull the first wire and bend the distal portion;
a shaft placed inside the operation portion;
a main pully configured to guide the first wire and configured to rotate around the shaft; and
a first sheath inserted into the insertion portion and receiving the first wire, the first sheath configured to guide the first wire toward the shaft.

13. The insertion device according to claim 12, further comprising a first guide configured to guide the first wire from the first sheath toward a circumference of the main pully.

14. The insertion device according to claim 13, wherein the first sheath is configured to guide the first wire such that a friction generated between the first wire and the first sheath is reduced.

15. The insertion device according to claim 12, further comprising:
a second wire inserted into the insertion portion, the second wire having a first end and a second end, wherein the first end of the second wire is connected to the distal portion and wherein the second wire is pulled by the operation portion; and
a second sheath inserted into the insertion portion and receiving the second wire, the second sheath configured to guide the second wire toward the shaft.

16. The insertion device according to claim 15, further comprising:
a first guide configured to guide the first wire from the first sheath toward a first circumference of the main pully; and
a second guide configured to guide the second wire from the second sheath toward a second circumference of the main pully.

17. The insertion device according to claim 16, wherein the first guide and the second guide are each a pulley.

18. The insertion device according to claim 15, wherein a longitudinal axis of the first sheath is parallel to a longitudinal axis of the second sheath.

19. The insertion device according to claim 15, further comprising a first guide configured to guide the first wire from the first sheath toward a first circumference of the main pully and configured to guide the second wire from the second sheath toward a second circumference of the main pully.

20. The insertion device according to claim 19, wherein a longitudinal axis of the first sheath is parallel to a longitudinal axis of the second sheath.
